(19) United States
(12) Patent Application Publication
(10) Pub. No.: US 2024/0366901 A1
(43) Pub. Date: Nov. 7, 2024

(54) Device to Hold a Nasal Cannula in Place without Irritation or Compressing the Cannula Tube and Method of Using

(71) Applicants: Steven Lee Sugarek, Houston, TX (US); Chester Alan Sugarek, Freeport, TX (US)

(72) Inventors: Steven Lee Sugarek, Houston, TX (US); Chester Alan Sugarek, Freeport, TX (US)

(21) Appl. No.: 18/831,054

(22) Filed: May 21, 2024

(51) Int. Cl. A61M 16/06; A61M 16/00

(52) U.S. Cl. CPC A61M 16/0672; A61M 16/0003; A61M 2205/0216; A61M 2209/088; A61M 2210/0618

(57) ABSTRACT

Disclosed is an elastomeric device and method of using to securely position a nasal cannula that is fitted to the cannula tubing between the ear and the head. The device comprises a flange and body with a cut though both. The device is positioned with the flange end located behind the ear facing the front and contacting the head. The inside diameter of the device is measurably smaller than the outside diameter of the cannula tube. The cut permits the device to be attached to the tube at point of use. Once attached the device is adjusted by the wearer to a position where the flange contacts the back of the wearer's ear and side of the head with enough tension to keep the nasal cannula in a preferred position without having to cinch a bolo slide tightly and uncomfortably.

### CROSS-REFERENCES RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### REFERENCE TO A “SEQUENCE LISTING”

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

This invention relates to a device to hold in place a nasal cannula in the nasal cavities of a human using frictional contact between the device and a wearer's skin behind the ears without irritating the user or compressing the cannula tubing.

This invention further relates to a method of using the device to hold a nasal cannula in the nasal cavities of a human.

### 2. The Current State of the Art

A nasal cannula provides a user supplemental oxygen or oxygen therapy through the nose. Nasal cannula is more comfortable and practical than a face mask and is best for long-term use. The oxygen is supplied by a thin, flexible tube that goes around the user's ears and into the nose. There are two prongs that go inside the nostrils to deliver the oxygen. The tube is connected to an oxygen supply, which may be piped if supplied in a hospital setting, or an oxygen tank or wearable oxygen concentrator if portable.

Certain health conditions, especially respiratory ailments, make it hard for a user to receive enough oxygen. In these cases, receiving supplemental oxygen through a nasal cannula may be necessary.

A nasal cannula may be required at any stage of life. Newborns may need one if their lungs are underdeveloped or having breathing difficulties at birth. Cannula may also be used if an adult is traveling to an area with higher altitudes where oxygen levels are lower.

Supplemental oxygen may be needed short or long term. Some people need it for the rest of their lives, while others only while recovering from an illness or during a specific need.

The amount of supplemental oxygen may vary depending on need. Nasal cannula may be high-flow or low-flow. A low-flow up to about 6 liters of oxygen per minute and a high-flow nasal up to about 60 liters of oxygen per minute. The range of flow rates required tubes of different internal diameters; larger for higher flow rates.

The use of a nasal cannula begins with selecting the correct tubing diameter based on oxygen flow rate. The tubes for a low-flow nasal cannula range from 3 mm to 7 mm outside diameter. The prongs should be long enough to enter the nostrils fully but not so wide to occlude them. The nasal cannula tubing is then connected to the oxygen source and set at the desired flow rate in liters per minute.

The prongs are placed in the nostrils, curved downward if the prongs are curved. If the tubing has a tab below the prongs, it should rest on the upper lip. The tubing is placed over the ears like a pair of eyeglasses. The tubes are then brought down behind the ears and underneath the chin to the front of the chest. If the tubes have an adjustable slide, they are then secured under the chin by moving the adjustable slide so the tubing is snug but not too tight.

One problem with cannula tubes currently available in the marketplace is that the tubes must be tightly secured by a bolo slide at the front of the chest to hold the prongs in the nasal cavities. The required tightness caused by the slide frequently irritates the ears or crimps the tubes resulting in obstruction of oxygen flow. The bolo slide may not remain in place allowing the nasal prongs to fall away from the nose. Another problem is that devices for securing the cannula cannot be placed around the tubes after they are fitted around the ears.

The invention described here overcomes these problems.

### 3. Description of the Related Art Including Information Disclosed Under 37 C.F.R. 1.97 and 1.98

There are numerous patents and published patent application in the field of the invention disclosed here for keeping a nasal cannula in place while limiting irritation, but none individually anticipate, nor one or more taken together, render the invention obvious. No U.S. patents or U.S. patent applications, not otherwise issuing as a patent, are relevant here as prior art in the field of devices that hold in place a nasal cannula using frictional contact between the device and the tube and a wearer's skin without irritation or compressing the cannula tubing or disclose a method of using such the device.

Prior art restraints for nasal cannula are commonly by straps passing over and/or behind the head or devices that fit over the tubes and behind the ears. They relieve some of the weight of the cannula tube that would normally press on the ears or work to hold the cannula tubes in place. U.S. Patent Appl. No. US 2007/0056590, Wolfson, I. A., Holder for Nasal Cannula; U.S. Patent Appl. No. US 2011/0203595, Hashemich, J., Cannula Security Piece; U.S. Patent Appl. No. US 2013/0146064, Dryden, H., Nasal Cannula Support Device; U.S. Pat. No. 4,836,200, Clark, R. D. and Egan, S. D., Oxygen Tube Support Strap; U.S. Pat. No. 6,536,436, McGlothen, R., Strap for Nasal Cannula; U.S. Pat. No. 7,735,490, Rinaldi, T. L., Adjustable Nasal Cannula Apparatus and Method of Use; U.S. Pat. No. 10,261,339, Sugarek, S., Skin Contact Reduction Device Eyewear and Other Human Ear and Temple Resting Equipment.

Others rely on covering the cannula tubes which pass over the ears in a soft or compressible material: U.S. Patent Appl. No. US 2022/0007752, Sason, R., Ear Protector Hook; U.S. Pat. No. 4,699,139, Marshall, M. F. and Kislow, N. C., Nasal Cannula Assembly with Patient Comfort Pad; U.S. Pat. No. 4,949,733, Sampson, R. D., Nasal Oxygen Cannula Pad; U.S. Pat. No. 5,025,805, Nutter, B., Nasal Cannula Assembly.

Still others rely on structures or clips to support the cannula tubes to relieve pressure on the ears. U.S. Pat. No. 8,701,669, Jackman, R. L. and Anderson, K. J., Nasal Cannula Positioning Device; U.S. Pat. No. 11,491,295, Quechuleno, C., Nasal Cannula Clip System and Method.

### BRIEF SUMMARY OF THE INVENTION

Nasal cannula is typically held in place by positioning the cannula tubes around a user's ears, running the tubes down to the user's front, and resting them on the upper part of the chest. If the cannula runs behind on the back side of the neck and down, a user can lay on it and deform the tubing. The retainers work better if the tubing is installed in the normal position, to run down the user's front chest area.

Other than positioning the tubes around the ears, nasal cannula in use today is fitted without a positioner of any kind. The nosepiece prongs are primarily held in position by tensile force created by draping the tubes over the ears creating frictional contact between the ears and head, pulling the tubes tight in front of the chest, and securing them with a bolo slide. Nevertheless, the cannula tends to fall out of position when the head is turned side-to-side or up-and-down. It is well known that individuals under hospital care are often bed-ridden and may be left alone for extended periods of time. At these times, if the cannula falls out of the nose, the loss of supplemental oxygen can create serious health problems for the user, and even death.

Cannula skin contact reduction devices in current use must be installed on the cannula tubing at the manufacturing plant. Damage can occur to the tubing because skin contact reduction devices in current use may constrict the tubing over time.

To keep the nasal nosepiece prongs inserted in a user's nostrils, the device disclosed here can be fitted to any nasal cannula tube at the point of use. The dimensions of the device disclosed here and its embodiments are gauged to the outside diameter of the tube to which it is to be fitted, thereby useful for a wide range of tubing sizes.

One side of the body and flange of the device is sliced through longitudinally in various patterns to permit it to be uncurled and fitted around the cannula tube at the point of use. The cut reduces the structural integrity of the device itself when positioned around the tube. The reduction in structural integrity permits the device to deform slightly in use minimizing crimping of the tube and skin irritation.

When loading is applied to the flange, it flexes back along the barrel applying a small amount of pressure on the tubing, therefore eliminating slippage. This effect with the natural friction that is found when the device contacts the tubing ensures fit without slippage. It further minimizes or eliminates the need for a bolo slide.

These features with other technological improvements, which will become subsequently apparent, reside in the details as more fully described hereafter and claimed, reference being had to the accompanying drawings forming a part hereof.

### BRIEF DESCRIPTION OF THE SEVERAL VIEWS OF THE DRAWINGS

The present application is more fully understood by reference to the following figures, which are for illustrative purposes only. The figures are not necessarily drawn to scale and elements of similar structures or functions are represented by like reference numerals for illustrative purposes throughout the figures. The figures are only intended to facilitate the description of the various embodiments described herein. The figures do not describe every aspect of the teachings disclosed herein and do not limit the scope of the claims.

FIG. 1 illustrates the preferred embodiment of the device.

FIG. 2 shows the preferred embodiment of the device in cross-section.

FIG. 3 depicts the preferred device in place around a cannula tube.

FIG. 4 pictures a second embodiment.

FIG. 5 illustrates a third embodiment.

FIG. 6 shows a fourth embodiment.

FIG. 7 depicts a fifth embodiment.

FIG. 8 portrays a sixth embodiment.

FIG. 9 pictures a seventh embodiment.

FIG. 10 illustrates an eighth embodiment.

FIG. 11 shows a ninth embodiment.

FIG. 12 pictures the initial placement of the nasal cannula in the nares and the cannula tube over the left ear.

FIG. 13 illustrates further positioning of the nasal cannula tube over the left ear prior immediately prior to placing the preferred embodiment over the tube and behind the ear.

FIG. 14 shows opening the preferred embodiment along the radial cut on the top of the flange and the longitudinal spiral cut and placing it over the tube.

FIG. 15 depicts the preferred embodiment surrounding the cannula tube prior to final positioning behind the ear.

FIG. 16 portrays the preferred embodiment in final position.

### DETAILED DESCRIPTION OF THE INVENTION

### 1. Glossary of Terms Used in the Disclosure

The word “tube” or the phrases “cannula tube” or “cannula tubes” refer to the two flexible tubes with each of their ends connected to the nasal cannula prongs. The opposite ends of the tubes are further connected to a parallel wye connector known to a person having ordinary skill in the art which connects them to an oxygen supply.

The phrases “measurably smaller” or “measurably larger” mean one dimension is sufficiently less or more than another such that the difference may be determined by a measurement device capable of resolving at least 0.1 millimeter.

2. LIST OF REFERENCE NUMBERS.

| Number | Description |
|---|---|
| 100 | Preferred embodiment |
| 102 | Body |
| 104 | Flange |
| 106 | Hole in body |
| 108 | Cut encircling the body |
| 110 | Hole in flange |
| 112 | Cut extending radially through the flange |
| 114 | Point where the hole in the body meets the hole in the flange |
| 300 | Tube |
| 302 | Space between tube and flange |
| 400 | Second embodiment |
| 402 | Longitudinal cut along the length of the body |
| 500 | Third embodiment |
| 502 | Cut partially encircling the body and extending from the bottom |
| | of the body to midway along the length of the body |
| 504 | Point midway from the bottom of the body to a point at which |
| | the body meet the flange |
| 506 | Cut partially encircling the body and extending from midway |
| | along the length of the body to the point at which the cut |
| | meets the flange |
| 600 | Fourth embodiment |
| 602 | Beveled hole in the flange |
| 604 | Point where the beveled hole meets the top of the flange |
| 606 | Point where the beveled hole meets the hole in the body |
| 700 | Fifth embodiment |
| 800 | Sixth embodiment |
| 900 | Seventh embodiment |
| 1000 | Eighth embodiment |
| 1100 | Ninth embodiment |
| 1200 | User |
| 1202 | User's ear |
| 1204 | Nasal cannula |
| 1206 | Parallel wye connector |
| 1208 | Oxygen supply tube |
| 1210 | Second tube |
| 1212 | Bolo slide |

### 3. DETAILED DESCRIPTION OF THE PREFERRED AND OTHER EMBODIMENTS OF THE DEVICE

### (a) Detailed Description of the Preferred Embodiment

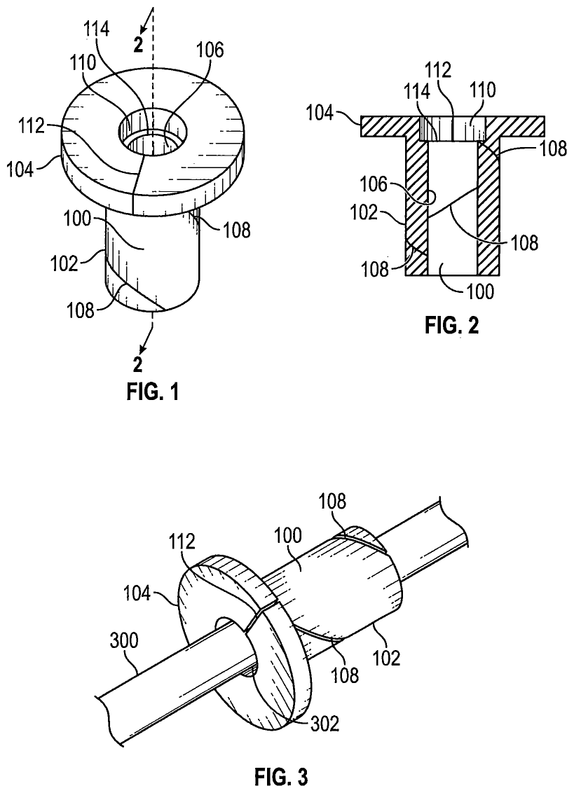

FIG. 1 shows the preferred embodiment 100. It comprises a body 102 and flange 104. 102 and 104 are made of a soft and pliable food grade moldable silicone-based polymer. 102 and 104 are indistinguishably connected to form one piece with coinciding center-lines. The center of hole 106 is located nominally at the center-line of 102 and extends uniformly from the bottom of 102 to 114, the place where 106 first contacts hole 110. From 114 to the top of flange 104, the center of hole 110 is located nominally at the center-line of flange 104.

The disclosed functionality of 100 requires that its dimensions be determined by the outside diameter of tube 300, depicted in FIG. 3, to which it is to be attached. The outside diameter of 102 is nominally 1.6 to 6 times the outside diameter of the tube 300, preferably 3 times. The inside diameter of 106 is nominally 0.75 to 1.0 times the outside diameter of 300; preferably 0.95 times. The inside diameter of hole 110, is nominally 1.0 to 1.10 times the outside diameter of 300, preferably 1.05 times. The length of 102 as measured from the end opposite flange 104 to the point at which it first contacts 104 is nominally 1 to 10 times the outside diameter of 300, preferably 5 times. The outside diameter of 104 is nominally 4 to 12 times the outside diameter of 300, preferably 6 times. The thickness of 104 as measured from bottom-to-top, is nominally 0.2 to 4 times the outside diameter of 300, preferably equal.

FIG. 1 also shows a cut 108 made through the side of 102 from outside to inside nominally tracking 180 to 720 degrees from the bottom of 102 to the point where 102 first contacts 104, preferably 360 degrees. At the point at which cut 108 reaches the bottom of 104, cut 112 is made radially from 110 through and to the outside of 104.

FIG. 2 is a cross-sectional view of 100. It depicts in greater detail inside diameter at 110 is measurably larger than the same of 106. It also shows 106 extending from the bottom of 102 to the point where 102 meets 104 at 114. 110 extends from 114 through to the top of 104.

FIG. 3 shows 100 in place around tube 300. It further shows that when 100 is placed around 300, the two surfaces formed by cuts 108 and 112 are slightly separated. The slight separation results in space depicted at 302 between the inside of 104 and 300. The space at 302 formed between 300 and 104 is more than what it would otherwise be if the inside diameter of 110 was not larger than the same of 106.

### (b) Detailed Descriptions of the Other Embodiments

Eight additional embodiments are disclosed and depicted in FIGS. 4 to 11. Many of the structures and items disclosed in 100 are duplicated in embodiments 400 to 1100.

Items 102, 104, and 106 are duplicated in FIGS. 4-11.

Item 108 is duplicated in FIGS. 6 and 9.

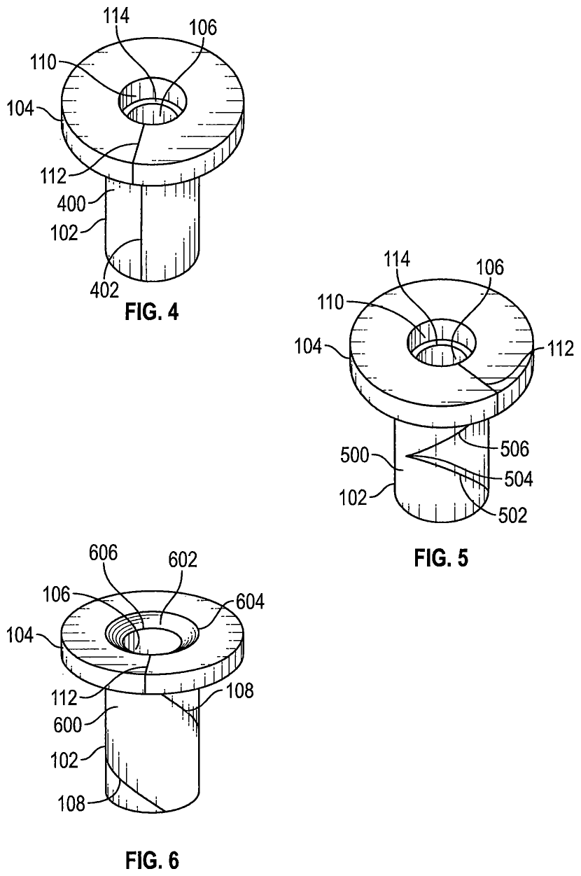

Items 110 and 114 are duplicated in FIGS. 4 and 5.

Item 112 is duplicated in FIGS. 4-11.

FIG. 4 shows second embodiment 400. FIG. 4 shows 402 is a cut made through the side of 102 from outside to inside coursing longitudinally from the bottom of 102 to the point where 402 first contacts 104. At the point at which 402 reaches the bottom of 104, 112 is made radially from the inside of 110 through and to the outside of 104.

FIG. 5 shows third embodiment 500. FIG. 5 shows cut 502 made through the side of 102 from outside to inside nominally tracking 90 to 360 degrees, preferably 180 degrees, from right-to-left from the bottom of 102 to the point where 502 first contacts 504, the nominal longitudinal midpoint of 102. From point 504, cut 506 is made through the side of 102 from outside to inside nominally tracking 90 to 360 degrees, preferably 180 degrees, from left-to-right from 504 to the point where 506 first contacts 104. At the point at which cut 506 reaches the bottom of 104, cut 112 is made radially from 110 to the outside of 104.

FIG. 6 shows fourth embodiment 600. In this embodiment, the center of hole 106 is nominally located at the center-line of 102 and extends uniformly from the bottom of 102 to a point where 102 first contacts 104 at 606. From 606 to the top of 104, the center of beveled hole 602 is nominally located at the center-line of 104.

The inside diameters of holes 106 and 602 are nominally the same where they meet at 606. The inside diameter of 602 at 604 is nominally the same as the outside diameter of 102. The inside diameter of 602 uniformly increases from 606 to 604. The diameter of 602 at 604 is nominally the same as the outside diameter of 102.

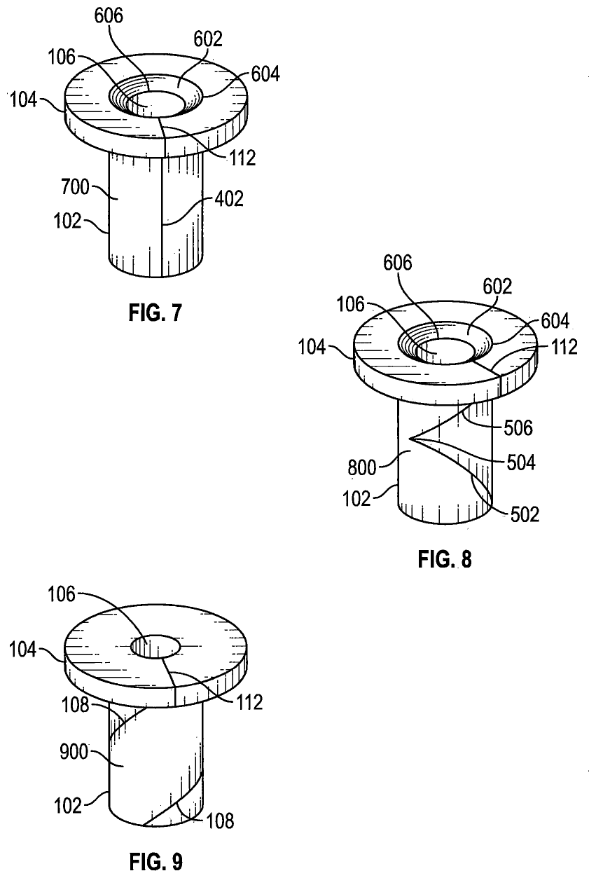

FIG. 7 shows fifth embodiment 700. It depicts items previously disclosed but combined to form a fifth embodiment.

FIG. 8 shows sixth embodiment 800. It depicts items previously disclosed but combined to form a sixth embodiment.

FIG. 9 shows seventh embodiment 900. It depicts items previously disclosed but combined to form a seventh embodiment.

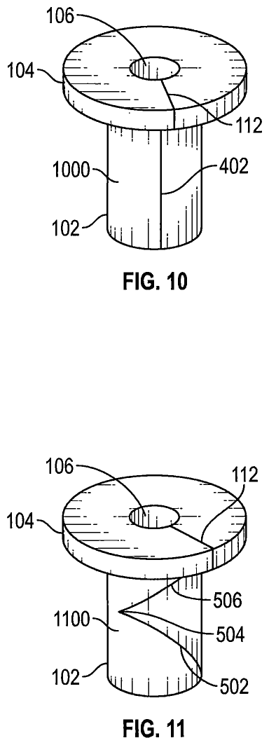

FIG. 10 shows eighth embodiment 1000. It depicts items previously disclosed but combined to form an eighth embodiment.

FIG. 11 shows ninth embodiment 1100. It depicts items previously disclosed but combined to form a ninth embodiment.

### 4. Detailed Description of the Method of Using the Preferred Embodiment of the Device

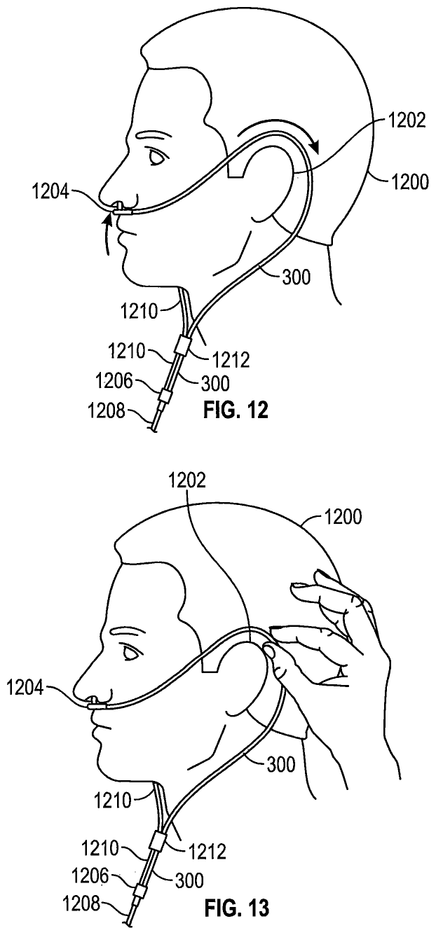

FIG. 12 depicts the first step in using devices 100, 400, 500, 600, 700, 800, 900, 1000, or 1100. Oxygen supply tube 1208 is fitted to the upstream end of parallel wye connector 1206. Tubes 300 and 1210 and connected to the downstream end of 1206.

Tubes 300 and 1210 are then coursed through bolo slide 1212 and then attached to the left and right ends of nasal cannula 1204 with enough length for 1210 to fit loosely over the right ear and 300 over the left ear. Nasal cannula 1204 is fitted comfortably in the nose. 1206 and 1208 are then laid on the chest if user 1200 is laying down or hung loosely in front of the chest if 1200 is standing.

FIG. 13 shows the second step. 300 is lifted lightly away from ear 1202. Two of identical devices 100, 400, 500, 600, 700, 800, 900, 1000, or 1100 are selected, one to be fitted around tube 300 and another around 1210.

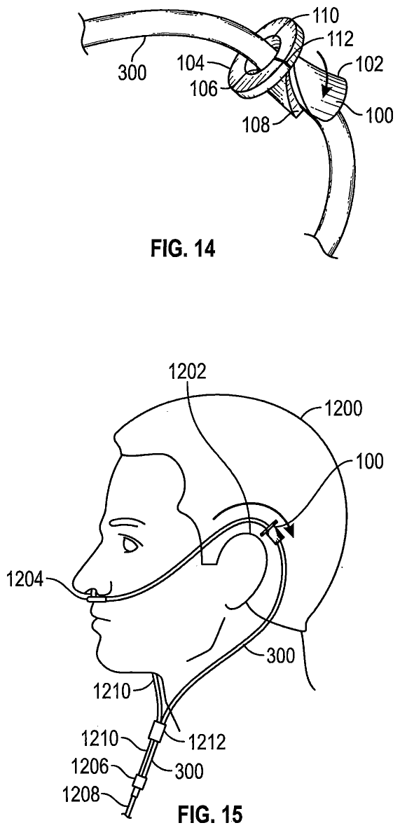

FIG. 14 shows the third step. One of 100, 400, 500, 600, 700, 800, 900, 1000, or 1100, is selected. In this depiction, body 102 of 100 is uncurled at cuts 108 and 112 enough to fit 100 over 300 with flange 104 towards the front of user 1200. 100 is then released causing it to relax and snuggly fit around 300. FIG. 14 shows that when 100 is in its relaxed position, flange 104 does not contact 300 due to the enlarged inside diameter of 110.

FIG. 15 shows final positioning of 100. 300 is pulled slightly towards the back of the head of 1200 and laid on the skin between ear and the side of the head. Once 1204 and 300 are at or near proper positions, 100 is uncurled slightly and slid over 300 to a point behind ear 1202. If necessary, bolo slide 1212 is cinched comfortably under the chin of 1200.

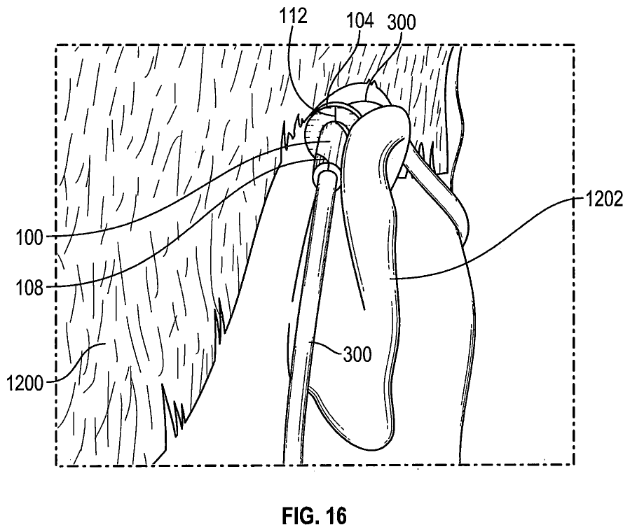

FIG. 16 shows 100 in final position around 300 and between the head of 1200 and ear 1202 with flange 104 pointing towards the face. The position is the same for 400, 500, 600, 700, 800, 900, 1000, or 1100. 300 courses around the ear to the front of the face of 1200.

Persons having ordinary skill in the art of preventing irritation of the skin using nasal cannula will understand that the device and method of using the device described in the preferred embodiment can vary and remain within the invention herein described. Variations obvious to those persons skilled in the art are included in the invention.

This written description uses examples to disclose the invention, including the preferred embodiment, and to enable a person having ordinary skill in the relevant art to practice the. invention, including making and using any of the devices disclosed here or and performing any incorporated methods. The patentable scope of the invention is defined by the claims, and may include other examples that occur to those persons of ordinary skill in the art. Such other examples are intended to be within the scope of the claims if they have structural elements that do not differ from the literal language of the claims, or if they include equivalent structural elements with insubstantial differences from the literal language of the claims.

Further, multiple variations and modifications are possible in the embodiments of the invention described here. Although a certain illustrative embodiment of the invention has been shown and described here, a wide range of modifications, changes, and substitutions is contemplated in the foregoing disclosure. In some instances, some features of the present invention may be employed without a corresponding use of the other features. Accordingly, it is appropriate that the foregoing description be construed broadly and understood as being given by way of illustration and example only, the spirit and scope of the invention being limited only by the appended claims.

We claim:

1. Claim 1. A device of a moldable, soft, and pliable material to securely hold nasal cannula prongs in a nasal cavity of a human by attachment to a cannula tube, said cannula tube having a first outside diameter, comprising;
a. a body comprising a first solid cylinder having a length and a second outside diameter;
(1) said length greater than said second outside diameter;
b. a flange comprising a second solid cylinder having a thickness and a third outside diameter;
(1) said thickness less than said third outside diameter;
c. the second outside diameter is less than the third outside diameter;
d. said body having a first longitudinal centerline;
e. said flange having a second longitudinal centerline;
f. the flange indistinguishably and permanently joined to one end of the body so that said first longitudinal centerline and said second longitudinal centerline are coincident;
g. a first hole passing through the body with centerline equal to the first longitudinal center line;
(1) said first hole has a first inside diameter equal to or measurably smaller than said first outside diameter;
h. a second hole passing through the flange with centerline equal to the second longitudinal centerline;
(1) said second hole has a second inside diameter equal to or measurably larger than the first outside diameter;
i. centerlines of the first hole and the second hole are coincident; and
j. a cut extending through the side of the body and the flange.

2. A device of a moldable, soft, and pliable material to securely hold nasal cannula prongs in a nasal cavity of a human by attachment to a cannula tube, said cannula tube having a first outside diameter, comprising;
a. a body comprising a first solid cylinder having a length and a second outside diameter;
(1) said length greater than said second outside diameter;
b. a flange comprising a second solid cylinder having a thickness and a third outside diameter;
(1) said thickness less than said third outside diameter;
c. the second outside diameter is less than the third outside diameter;
d. said body having a first longitudinal centerline;
e. said flange having a second longitudinal centerline;
f. the flange indistinguishably and permanently joined to one end of the body so that said first longitudinal centerline and said second longitudinal centerline are coincident;
g. a first hole passing through the body with centerline equal to the first longitudinal center line;
(1) said first hole has a first inside diameter equal to or measurably smaller than said first outside diameter;
h. a second hole passing through the flange with centerline equal to the second longitudinal centerline;
(1) said second hole having a second inside diameter equal to or measurably larger than the first outside diameter where the flange joins the body and a third inside diameter equal to the second outside diameter farthest from where the flange joins the body;
(2) said second inside diameter increases uniformly through the thickness to said third inside diameter;
i. centerlines of the first hole and the second hole are coincident; and
j. a cut extending through the side of the body and the flange.

3. The device in claims 1 and 2 wherein;
a. said second outside diameter is 1.6 to 6 times said first outside diameter;
b. said third outside diameter is 4 to 12 times the first outside diameter;
c. said length is 1 to 10 times the first outside diameter;
d. said thickness is 0.2 to 4 times the first outside diameter;
e. said first inside diameter is 0.75 to 1.0 times the first outside diameter; and
f. said second inside diameter is 1.0 to 1.10 times the first outside diameter.

4. The device in claims 1 and 2 wherein;
a. said second outside diameter is 3 times said first outside diameter;
b. said third outside diameter is 6 times the first outside diameter;
c. said length is 5 times the first outside diameter;
d. said thickness is equal to the first outside diameter;
e. said first inside diameter is 0.95 times the first outside diameter; and
f. said second inside diameter is 1.05 times the first outside diameter.

5. The device in claims 1 and 2 wherein said cut courses in a spiral of 180 to 720 degrees from an end of said body farthest from said flange to the flange and then radially through the flange.

6. The device in claims 1 and 2 wherein said cut courses in a spiral of 360 degrees from an end of said body farthest from said flange to the flange and then radially through the flange.

7. The device in claims 1 and 2 wherein said cut courses in a spiral clockwise 90 to 360 degrees from an end of said body farthest from said flange to a longitudinal midpoint of the body and then spirally counterclockwise 90 to 360 degrees from said longitudinal midpoint to the flange and then radially through the flange.

8. The device in claims 1 and 2 wherein said cut courses in a spiral clockwise 180 degrees from an end of said body farthest from said flange to a longitudinal midpoint of the body and then spirally counterclockwise 180 degrees from said longitudinal midpoint to the flange and then radially through the flange.

9. The device in claims 1 and 2 wherein said cut extends longitudinally from an end of said body farthest from said flange to the flange and then radially through the flange.

10. The device in claims 1 and 2 wherein said moldable, soft, and pliable material is a food grade silicone-based polymer.

11. A method to securely hold nasal cannula prongs in a nasal cavity of a human by attaching a device to a cannula tube, said cannula tube having a first outside diameter, comprising;
a. selecting said device comprising;
(1) a body comprising a first solid cylinder having a length and a second outside diameter;
(i) said length greater than said second outside diameter;
(2) a flange comprising a second solid cylinder having a thickness and a third outside diameter;
(i) said thickness less than said third outside diameter;
(3) the second outside diameter is less than the third outside diameter;
(4) said body having a first longitudinal centerline;
(5) said flange having a second longitudinal centerline;
(6) the flange indistinguishably and permanently joined to one end of the body so that said first longitudinal centerline and said second longitudinal centerline are coincident;
(7) a first hole passing through the body with centerline equal to the first longitudinal center line
(i) said first hole has a first inside diameter equal to or measurably smaller than said first outside diameter;
(8) a second hole passing through the flange with centerline equal to the second longitudinal centerline;
(i) said second hole has a second inside diameter equal to or measurably larger than the first outside diameter;
(9) centerlines of the first hole and the second hole are coincident; and
(10) a cut extending through the side of the body and the flange;
b. fitting said nasal cannula prongs loosely in said nasal cavity;
c. stringing said cannula tube over each ear
d. bringing the cannula tube to front of said human;
e. uncurling the device at said cut enough to fit the device around the cannula tube;
f. orienting the device so the flange faces said front of the human;
g. placing the device around the cannula tube behind said ear;
h. releasing the device to achieve a rest configuration around the tube; and
i. positioning the device along the cannula tube.

12. A method to securely hold nasal cannula prongs in a nasal cavity of a human by attaching a device to a cannula tube, said cannula tube having a first outside diameter, comprising;
a. selecting said device comprising;
(1) a body comprising a first solid cylinder having a length and a second outside diameter;
(i) said length greater than said second outside diameter;
(2) a flange comprising a second solid cylinder having a thickness and a third outside diameter;
(i) said thickness less than said third outside diameter;
(3) the second outside diameter is less than the third outside diameter;
(4) said body having a first longitudinal centerline;
(5) said flange having a second longitudinal centerline;
(6) the flange indistinguishably and permanently joined to one end of the body so that said first longitudinal centerline and said second longitudinal centerline are coincident;
(7) a first hole passing through the body with centerline equal to the first longitudinal center line;
(i) said first hole has a first inside diameter equal to or measurably smaller than said first outside diameter;
(8) a second hole passing through the flange with centerline equal to the second longitudinal centerline;
(i) said second hole having a second inside diameter equal to or measurably larger than the first outside diameter where the flange joins the body and a third inside diameter equal to the second outside diameter farthest from where the flange joins the body;
(ii) said second inside diameter increases uniformly through the thickness to said third inside diameter;
(9) centerlines of the first hole and the second hole are coincident; and
(10) a cut extending through the side of the body and the flange;
b. fitting said nasal cannula prongs loosely in said nasal cavity;
c. stringing said cannula tube over each ear
d. bringing the cannula tube to front of said human;
e. uncurling the device at said cut enough to fit the device around the cannula tube;
f. orienting the device so the flange faces said front of the human;
g. placing the device around the cannula tube behind said ear;
h. releasing the device to achieve a rest configuration around the tube; and
i. positioning the device along the cannula tube.

13. Claim 13. The method in claims 11 and 12 wherein;
a. said second outside diameter is 1.6 to 6 times said first outside diameter;
b. said third outside diameter is 4 to 12 times the first outside diameter;
c. said length is 1 to 10 times the first outside diameter;
d. said thickness is 0.2 to 4 times the first outside diameter;
e. said first inside diameter is 0.75 to 1.0 times the first outside diameter; and
f. said second inside diameter is 1.0 to 1.10 times the first outside diameter.

14. The method in claims 11 and 12 wherein;
a. said second outside diameter is 3 times said first outside diameter;
b. said third outside diameter is 6 times the first outside diameter;
c. said length is 5 times the first outside diameter;
d. said thickness is equal to the first outside diameter;
e. said first inside diameter is 0.95 times the first outside diameter; and
f. said second inside diameter is 1.05 times the first outside diameter.

15. The method in claims 11 and 12 wherein said cut courses in a spiral of 180 to 720 degrees from an end of said body farthest from said flange to the flange and then radially through the flange.

16. The method in claims 11 and 12 wherein said cut courses in a spiral of 360 degrees from an end of said body farthest from said flange to the flange and then radially through the flange.

17. The method in claims 11 and 12 wherein said cut courses in a spiral clockwise 90 to 360 degrees from an end of said body farthest from said flange to a longitudinal midpoint of the body and then spirally counterclockwise 90 to 360 degrees from said longitudinal midpoint to the flange and then radially through the flange.

18. The method in claims 11 and 12 wherein said cut courses in a spiral clockwise 180 degrees from an end of said body farthest from said flange to a longitudinal midpoint of the body and then spirally counterclockwise 180 degrees from said longitudinal midpoint to the flange and then radially through the flange.

19. The method in claims 11 and 12 wherein said cut extends longitudinally from an end of said body farthest from said flange to the flange and then radially through the flange.

20. The method in claims 11 and 12 wherein said device is formed from a moldable, soft, and pliable food grade silicone-based polymer.

21. A device of a moldable, soft, and pliable material to securely hold nasal cannula prongs in a nasal cavity of a human by attachment to a cannula tube, said cannula tube having a first outside diameter, comprising;
a. a body comprising a first solid cylinder having a length and a second outside diameter;
(1) said length is 5 times said first outside diameter and said second outside diameter is 3 times the first outside diameter;
b. a flange comprising a second solid cylinder having a thickness and a third outside diameter;
(1) said thickness is equal to the first outside diameter and said third outside diameter is 6 times the first outside diameter;
c. said body having a first longitudinal centerline;
d. said flange having a second longitudinal centerline;
e. the flange indistinguishably and permanently joined to one end of the body so that said first longitudinal centerline and said second longitudinal centerline are coincident;
f. a first hole passing through the body with centerline equal to the first longitudinal center line;
(1) said first hole has a first inside diameter 0.95 times the first outside diameter;
g. a second hole passing through the flange with centerline equal to the second longitudinal centerline;
(1) said second hole has a second inside diameter 1.05 times the first outside diameter;
h. centerlines of the first hole and the second hole are coincident;
i. a cut extending through the side of the body and the flange;
(1) said cut courses in a spiral of 360 degrees from an end of the body farthest from the flange to the flange and then radially through the flange; and
j. said moldable, soft, and pliable material is a food grade silicone-based polymer.
